(19) United States
(12) Patent Application Publication
(10) Pub. No.: US 2011/0059137 A1
(43) Pub. Date: Mar. 10, 2011

(54) CHEMOKINE GENE-MODIFIED CELLS FOR CANCER IMMUNOTHERAPY

(75) Inventors: Scott Joseph Antonia, Land O'Lakes, FL (US); James J. Mule, Odessa, FL (US)

(73) Assignees: H. LEE MOFFITT CANCER CENTER AND RESEARCH INSTITUTUTE, INC, Tampa, FL (US); UNIVERSITY OF SOUTH FLORIDA, Tampa, FL (US)

(21) Appl. No.: 12/933,568

(22) PCT Filed: Mar. 19, 2009
(86) PCT No.: PCT/US09/37652

§ 371 Date: Nov. 23, 2010

Related U.S. Application Data
(60) Provisional application No. 61/038,531, filed on Mar. 21, 2008.
(60) Provisional application No. 61/039,641, filed on Mar. 26, 2008.

(51) Int. Cl. A61K 39/00; C12N 5/09; A61P 35/00; A61P 37/04

(52) U.S. Cl. 424/277.1; 435/347

(57) ABSTRACT

Described herein are methods of cancer immunotherapy, particularly compositions comprising genetically-modified cells that express macrophage colony stimulating factor (GM-CSF), CD40 ligand (CD40L), and chemokine C—C motif ligand 21 (CCL21), wherein the population of cells comprises bystander cells and target cancer cells, and methods of making these compositions and treating cancer using these compositions.

### CROSS REFERENCE TO RELATED APPLICATIONS

This application claims the benefit of U.S. Provisional Application Ser. No. 61/038,531, filed on Mar. 21, 2008, and U.S. Provisional Application Ser. No. 61/039,641, filed on Mar. 26, 2008. The entire contents of the foregoing are incorporated herein by reference.

### STATEMENT REGARDING FEDERALLY SPONSORED RESEARCH OR DEVELOPMENT

This invention was supported by grant no. CA071669 awarded by the National Institutes of Health. The government has certain rights in the invention.

### TECHNICAL FIELD

The technology described herein generally relates to methods of cancer immunotherapy. The technology more particularly relates to compositions of cancer vaccine comprising cells expressing cDNAs encoding GM-CSF, CD40 ligand, and CCL21, and methods of making these compositions and treating cancer using these compositions.

### BACKGROUND

Tumor cells possess multiple means of evading T cell-mediated rejection. This occurs despite the fact that there are tumor-associated antigens (TAAs) expressed by transformed cells but not by most normal cells (Rosenberg, Cancer J Sci Am 6 (Suppl 3):5200 (2000)), and despite the fact that T cells specific for TAAs are in fact present within cancer patients (Pittet et al., J Exp Med 190:705 (1999); Anichini et al., J Exp Med 190:651 (1999); Jager et al., Proc Natl Acad Sci USA 97:4760 (2000)). One mechanism whereby tumors escape immune-mediated destruction is by interfering with dendritic cells (DCs), which are potent antigen-presenting cells whose proper functioning is important in the induction of antigen-specific T cell responses (Banchereau et al., Nature 392:245 (1998)). Tumor-derived VEGF can interfere with the differentiation of DCs (Gabrilovich et al., Nat Med 2:1096 (1996)), as can IL-6 and MCS-F (Menetrier-Caux et al., Blood 92:4778 (1998)). Also, IL-10 is secreted by some tumors (Chen et al., Int J Cancer 56:755 (1994); Smith et al., Am J Pathol 145:18 (1994); Huang et al., Cancer Res 55:3847 (1995)), and this cytokine has been shown to interfere with DC function (Steinbrink et al., J Immunol 159:4772 (1997); Koch et al., J Exp Med 184:741 (1996); Steinbrink et al., Blood 93:1634 (1999)). Tumors are also capable of forcing DCs to undergo apoptosis (Pirtskhalaishvili et al., Br J Cancer 83:506 (2000); Esche et al., Clin Cancer Res 7 (3 Suppl):974s (2001); Kiertscher et al., J Immunol 164:1269 (2000)).

A number of clinical trials have demonstrated that vaccines can be used to produce anti-tumor immune responses in human lung cancer patients. The vaccines included WT1 peptide (Tsuboi et al., Microbiol Immunol 48:175 (2004)), MAGE-3 protein (Atanackovic et al., J Immunol 172:3289 (2004)), or UBE2V peptide (Harada et al., J Immunol 172:2659 (2004)) emulsified in immunologic adjuvants; lung tumor cell-pulsed (Hirschowitz et al., J Clin Oncol 22:2808 (2004)) or CEA peptide-pulsed DCs (Ueda et al., Int J Oncol 24:909 (2004)); and gene-modified autologous (Nemunaitis et al., J Natl Cancer Inst 96:326 (2004); Salgia et al., J Clin Oncol 21:624 (2003)) or allogeneic tumor cells (Raez et al., Cancer Gene Ther 10:850 (2003); Raez et al., J Clin Oncol 22:2800 (2004)). The latter approach involved patients with measurable disease, and tumor regressions were observed. Raez et al. treated 19 NSCLC patients (11 with adenocarcinoma) who were HLA A1 or A2 positive with an HLA A1- or A2-expressing allogeneic lung adenocarcinoma cell line that was transfected with the B7-1 gene. Five patients had stable disease and 1 developed a durable partial response (Raez et al., J Clin Oncol 22:2800 (2004)). Nemunaitis et al. treated 33 advanced-stage NSCLC patients with autologous tumor cells that were transfected with the GM-CSF gene. Three of these patients developed a durable complete response (Nemunaitis et al., J Natl Cancer Inst 96:326 (2004)). As a result of this trial, this GM-CSF-based vaccine was tested in a trial conducted by the Southwest Oncology Group (See, e.g., Neumanitis et al., Cancer Gene Therapy 13:555-562 (2006); and Neumanitis et al., J Control Release. 91 (1-2):225-31 (2003)).

A number of other GM-CSF based vaccines, designed to enhance DC function at a tumor vaccine site, are currently being tested in clinical trials. With these vaccines, autologous tumor cells (Simons et al., Cancer Res 57:1537 (1997); Chang et al., Hum Gene Ther 11:839 (2000); Simons et al., Cancer Res 59:5160 (1999); Soiffer et al., Proc Natl Acad Sci USA 95:13141 (1998); Kusumoto et al., Cancer Immunol Immunother 50:373 (2001)), allogeneic tumor cell lines (Jaffee et al., J Clin Oncol 19:145 (2001)), or bystander cell lines (Borrello et al., Hum Gene Ther 10:1983 (1999)) are transfected with the human GM-CSF gene. The GM-CSF-producing tumor cells, or bystander cells admixed with tumor cells, are injected into patients as the vaccine. The GM-CSF secreted at the vaccine site results in the recruitment and differentiation of DCs (Mach et al., Curr Opin Immunol 12:571 (2000); Nelson et al., Cancer Chemother Pharmacol 46 (Suppl):S67 (2000)), and the tumor cells serve as the source of TAAs that are processed by the DCs, which subsequently migrate to lymph nodes where they activate TAA-specific T cells.

### SUMMARY

At least in part, the present invention is based on the discovery that expression of chemokine C—C motif ligand 21 (CCL21) augments anti-tumor immune responses, and therefore, can improve the efficacy of cancer immunotherapy.

Thus, in one aspect, the invention provides populations of cells that have been genetically-modified to express (and secrete) exogenous macrophage colony stimulating factor (GM-CSF), to express exogenous CD40 ligand (CD40L) (and thus have CD40L on their cell surface), and to express (and secrete) exogenous chemokine C—C motif ligand 21 (CCL21), wherein the population of cells includes bystander cells and target cancer cells. Not all of the cells need express all of the genes, for example, in some embodiments, the bystander cells express GM-CSF and CD40L; the bystander cells can also express CCL21, or the target cancer cells express CCL21 (if there is more than one type of target cancer cell, e.g., two or more types of cancer cells, then only one or all of them can express CCL21).

In general, the bystander cells are major histocompatibility complex (MHC) negative; in some embodiments, the bystander cells are from the cell line K562.

The target cancer cells can include cells from a solid or hematopoietic-derived tumor. The cells can be from, e.g., one, two or more allogeneic cancer cell lines, or primary cancer cells, e.g., allogeneic or autologus (i.e., cells from a subject to whom the population of cells will ultimately be administered as a vaccine). In some embodiments, the target cancer cells include cells from two or more different cancer types or different cell lines, e.g., cells from two or more different human lung adenocarcinoma cell lines. Exemplary cell lines include NCI-H1944 and NCI-H2122, available from ATCC.

In some embodiments, the cells have been treated to reduce cell viability, e.g., in preparation for administration to a subject. In general, suitable treatments will induce apoptosis within a short period of time, causing the cells to die and break up.

In another aspect, the invention provides therapeutic compositions for inducing an immune response to a cancer in a subject, including a population of cells as described herein.

In yet a further aspect, the invention provides methods for preparing a population of cells for use in a therapeutic composition. The methods include providing a population of cells as described herein, and treating the cells to reduce cell viability.

Also included herein is the use of a population of cells as described herein in the manufacture of a medicament for the treatment of cancer, the use of a population of cells as described herein as a medicament, and the use of a population of cells as described herein for the treatment of a cancer.

In another aspect, the invention provides methods for treating a cancer in a subject, e.g., a non-human animal or a human. The methods include administering to the subject a therapeutically effective amount of a composition including a population of cells as described herein, e.g., cells that have been treated to reduce viability.

In some embodiments, the target cancer cells include cancer cells that are autologous to the subject to be treated (other types of cancer cells can also be included, e.g., from cell lines or from other subjects). In some embodiments, the target cancer cells include cells from a cancer of the same type as the cancer in the subject. In some embodiments, the target cancer cells include cells from a cell line made from cells of a cancer of the same type as the cancer in the subject. Mixtures of cell types can also be included.

In some embodiments, the composition is administered by a route of administration selected from the group consisting of: subcutaneous, intradermal and subdermal.

The methods described herein can also include administering one or more additional treatments to the subject, e.g., a known or conventional treatment for the cancer, e.g., chemotherapy, radiation, or surgery.

In some embodiments, the methods include administering one or more additional doses of the composition.

In some embodiments, the methods also include a step of identifying a subject having a cancer. In some embodiments, the methods also include monitoring the subject for one or more clinical parameters of cancer, e.g., one or more clinical parameters of cancer selected from the group consisting of: tumor growth, tumor regrowth and survival.

The methods and compositions described herein can be used in the treatment of cancers selected from the group consisting of: lymphoma, non-Hodgkin's lymphoma, leukemia, myeloma, glioma, neuroblastoma, lung cancer, kidney cancer, liver cancer, breast cancer, prostate cancer, gastric cancer, pancreatic cancer, colon cancer, soft tissue sarcoma, bone sarcoma and melanoma. Thus the populations of cells can be made using cells from any of these types of cancers, e.g., from primary cells or cell lines from any of these types of cancers.

Throughout the description and claims of the specification the word “comprise” and variations thereof, such as “comprising” and “comprises”, is not intended to exclude other additives, components, integers or steps.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, can control.

Other features and advantages of the invention can be apparent from the following detailed description and figures, and from the claims.

### BRIEF DESCRIPTION OF THE DRAWINGS

FIGS. 1A and 1B are bar graphs showing CCL21 expression of the Ad.CCL21-transduced H1944 cells. H1944 cells were infected with Ad.CCL21 at the indicated multiplicity of infection (MOI) (uninfected, 50:1, 100:1, 200:1). The cells were then treated as indicated. Frozen cells were thawed and the cells cultured in media for 48 hours (1A) or 72 hours (1B). Culture supernatants were collected and assayed by ELISA.

FIG. 2 is a bar graph showing that naïve T cells migrate in response to CCL21 secreted by Ad.CCL21-transduced H1944 cells. Naïve T cells were obtained from PBMC of a healthy donor using an untouched T cell isolation kit. Chemotaxis was assayed at 72 and 96 hours post transduction using Corning transwell plates.

FIG. 3 is a bar graph showing that CCL21 expression augments anti-tumor immune responses induced by GM.CD40L-transfected bystander cells. T cell-associated IL-2 secretion by lymph node (LN) cells increased in the presence of H1944-derived CCL21 over untransduced H1944 tumor cells when co-cultured with bystander cells.

### DETAILED DESCRIPTION

As described herein, the efficacy of cancer immunotherapy can be significantly increased by treatment with a cell-based vaccine including a mixture of bystander cells and a population of target cancer cells, wherein the cells express GM-CSF, CD40 ligand, and CCL21 following gene(s) transfer. Described herein are compositions including these cell-based vaccines, as well as methods for treating cancer, e.g., by eliciting an anti-tumor immune response in a subject, using the described cell-based vaccines.

As one theory, it is believed that, in the context of allogeneic tumor cell-based vaccine formulations, CCL21 and GM-CSF secretion at the vaccine site microenvironment recruits and differentiates professional antigen presenting cells (APCs) in the form of dendritic cells (DCs) that can be activated by their encounter with CD40 ligand on the surface of the bystander cells. Apoptotic bodies from the radiated tumor cells are taken up and processed by the DCs. CD40 ligation results in the activation of cross presentation of exogenous antigens taken up by DCs on MHC class I molecules, DCs at the vaccine sites can load shared tumor antigen derived peptides onto both MHC class I and II molecules. These activated and antigen-loaded DCs then migrate to the draining lymph nodes and activate tumor antigen specific T cells (CD4 and CD8) in the lymph nodes, as well as T cells that are recruited into the actual vaccine site by CCL21. These activated T cells then recirculate to metastatic sites and kill tumor cells.

A phase I trial testing bystander cells expressing GM-CSF and CD40L following gene(s) transfer admixed with autologous tumor cells as a vaccine in patients with a variety of solid tumors was completed. The vaccine was safe, and anti-tumor cell immune responses as well as clinical responses were induced.

### Cell-Based Vaccines

In general, the vaccines described herein are prepared by mixing a bystander cell line with a population of target cancer cells to formulate the final vaccine product. The cells express GM-CSF, CD40L, and CCL21 following gene(s) transfer. In some embodiments, the bystander cells express GM-CSF and CD40L following gene(s) transfer, and the cancer cells express CCL21 following gene transfer; where more than one type of cancer cell is used, one or more of the types can express CCL21 following gene transfer. In some embodiments, the bystander cells express GM-CSF, CD40L, and CCL21 following gene(s) transfer.

### Bystander Cell Line

Cancer cell lines that are MHC negative can be used as the bystander cell line. For example, Levitsky and colleagues (Borrello et al., Hum Gene Ther 10:1983 (1999)) have described the use of a vaccine in which a universal MHC-negative GM-CSF-producing “bystander cell” is mixed with irradiated tumor cells (antigen source). With the bystander vaccine approach, there is no need to genetically manipulate the autologous tumor cells. The parental cell line chosen for the “bystander cell line” was K562, a human erythroleukemia cell line, because it is MHC-negative (potentially decreasing the magnitude of allogeneic responses that could shorten the duration of GM-CSF production on repeated immunization), and it can be grown in suspension cultures (facilitating large-scale production required for clinical testing). This autologous tumor cell/universal bystander cell vaccine, called K562 Bystander GVAX®, was developed by the Johns Hopkins Cancer Center in collaboration with Cell Genesys, Inc., and has completed testing in phase I/II clinical trials in patients with multiple myeloma and AML (see, e.g., Dummer et al., Curr Opin Investig Drugs. 2(6):844-8 (2001); Gorin et al., Hematology Am Soc Hematol Educ Program. 2000:69-89).

In some embodiments, K562 cells are grown in suspension cultures and are maintained at 37° C. in a 5% CO₂ humidified environment in Iscove's medium supplemented with 10% fetal calf serum (FCS), 50 U/mL penicillin-streptomycin, 2 mM L-glutamine, and 50 mM 2-mercaptoethanol (complete medium).

Chiodoni et al. have extended the concept of using GM-CSF-based vaccines by transfecting the gene coding for CD40 ligand into tumor cells along with the GM-CSF gene in a murine model (Chiodoni et al., J Exp Med 190:125 (1999)). CD40 ligand is a potent activator of dendritic cells (Cella et al., J Exp Med 184:747 (1996)) that results in the upregulation of surface T cell costimulatory molecules and the increase in the secretion of cytokines (Peguet-Navarro et al., J Immunol 155:4241 (1995); Caux et al., J Exp Med 180:1263 (1994)). When both the GM-CSF and CD40 ligand genes were transfected into tumor cells, more mice transplanted with the tumor cells remained tumor free than mice transplanted with tumor cells that were transfected with the GM-CSF gene alone.

Brenner and colleagues tested a CD40 ligand-based tumor vaccine in a murine model of multiple myeloma (Dotti et al., Blood 100:200 (2002)). They used an approach similar to that of Levitsky and colleagues, utilizing a bystander cell strategy. They engineered a bystander cell line to express CD40 ligand and admixed these bystander cells with tumor cells as the source of tumor antigens. They found that this vaccine was very effective in protecting mice from a tumor challenge by recruiting and activating professional antigen-presenting cells at the vaccine site.

The transfection of a bystander cell line can be achieved using methods known in the art. See, e.g., Example 1, herein; as well as Dessureault et al., J Surg Res 125:173 (2005); Dessureault et al., Ann Surg Oncol 14(2):869 (2006); U.S. patent application Ser. Nos. 10/620,746 and 12/173,514.

### Target Cancer Cells

The population of target cancer cells provides the tumor antigens. Depending on the specific tumor type to be treated in a subject, the cells can be from a solid or hematopoietic-derived tumor. Tumors can be harvested surgically from subjects. The harvested tumors can be used freshly or cryopreserved for later use. A single cell suspension can be made by a combination of mechanical and enzyme dispersion techniques. For long-term storage, cancer cells can be frozen in a liquid nitrogen freezer.

In some embodiments, the target cancer cells are obtained from the subject to whom they can be delivered, i.e., autologous, or from another subject having the same type of cancer, i.e., allogeneic.

In some embodiments, the methods include obtaining a sample of a tumor in a subject to be treated using a method described herein, and detecting the presence of tumor-associated antigens (TAA) on cells of the tumor. Then, cells from a tumor in another subject, or from a combination of tumors in other subjects, can be chosen that express the same tumor-associated antigens. A number of tumor-associated antigens are known in the art, and methods for detecting them are well known. For example, several TAAs over-expressed in NSCLC cell lines have been identified. These include MAGE-1, 2, and 3, CEA, HER-2/neu, and WT-1. Characterization of 31 NSCLC lines showed that the majority tested express HER-2/neu (90%) and CEA (58%) on the cell surface. Two lung adenocarcinoma cell lines, NCI-H1944 and NCI-H2122, that together express HER-2/neu, CEA, GD-2, WT-1, and MAGE-1, -2, and -3 (Wroblewski et al., Lung Cancer 33:181 (2001)) can be used.

In some embodiments, the target cancer cells are obtained from one or more cell lines made from cells of a tumor that is from the same type of cancer that the subject has, e.g., one or more human non-small cell lung cancer (NSCLC) cell lines for use in a subject who has NSCLC. Cancer cell lines are known in the art, and numerous examples are commercially available, e.g., from the American Type Culture Collection (ATCC) (Manassas, Va.), which has over 1100 different tumor cell lines from a variety of cancer types and species. For example, HPAC for pancreatic cancer, CA-HPV-10 for prostate cancer, DLD-1 for colon cancer, TOV-21G for ovarian cancer, 786-O for kidney cancer, HepG2 for liver cancer, M059K for brain cancer, 8E5 for acute lymphoblastic leukemia, 1A2 for lymphoma, NCI-H929 for myeloma.

Chemokine C—C motif ligand 21 (CCL21)

CCL21 (also called Exodus, 6Ckine, or SLC) is a CC family chemokine capable of recruiting DCs and naïve T cells expressing CCR7. Homing of T cells to the lymph node is achieved by production of CCL21 in high endothelial venules (HEV) of the lymph node. Previously, the CCL21 (Ad.CCL21) cDNA has been transduced into DCs via recombinant adenovirus vectors with the ability to prime autologous T cells (Terando et al., Cancer Gene Ther 11:165 (2004)). Administration of irradiated CCL21-producing tumor cells can create an extranodal zone enabling DCs and T cells to interact in the presence of tumor antigen. The DC-T cell rich environment minimizes the requirement for DCs to migrate to lymph node regions prior to antigen presention. Several groups have demonstrated improved anti-tumor responses following intra-tumoral introduction of the CCL21 cDNA through transduced DCs in mouse models (Kirk et al., Cancer Res 61:2062 (2001); Yang et al., Clin Cancer Res 10:2891 (2004)). Combination of CCL21 production with costimulatory molecules has demonstrated synergistic antitumor effects (Hisada et al., Cancer Gene Ther 11:280 (2004)) and increases in IFN-γ-producing CD8⁺ T cells while inducing apoptosis in CD4⁺CD25⁺FoxP3⁺ regulatory T cells (Liu et al., J Immunol 178:3301 (2007)). In cell lines, CCL21 cDNA-transfected MCF-7 breast cancer can induce migration, antigen uptake, and presentation of human monocyte-derived DCs (Wu et al., Immunobiol 213:417 (2008)). Those DCs are also able to facilitate the generation of CD8⁺ T effector cells with the subsequent clearance of the MCF-7.

Exemplary nucleic acid sequences for CCL21 are NM_{—}002989.2 for human, NM_{—}001032855.1 for rhesus monkey, and NM_{—}001005151.1 for pig. Exemplary amino acid sequences for CCL21 are NP_{—}002980.1 for human, NP_{—}001028027.1 for rhesus monkey, and NP_{—}001005151.1 for pig.

CCL21 gene bearing adenovirus (Ad.CCL21) can be obtained from commercial resources. Adenoviral infection of cells can be performed with known methodology. In some embodiments, Ad.CCL21 can be added to cells, e.g., a suspension of target cancer cells, e.g., at an MOI of 1,000 to 100,000 pfu/cell, e.g. 10,000 pfu/cell, e.g., 50,000 pfu/cell. Cells are placed in a 37° C. incubator for 1 to 10 hours, e.g., 2 hours, e.g., 5 hours, to promote viral adsorbtion to cells. Following the incubation, cells are adjusted to 1×10⁶ to 1×10⁸ cells/mL, e.g., 1×10⁷ cells/mL, e.g., 3×10⁷ cells/mL, and returned to the incubator for 12 to 48 additional hours, e.g, 24 hours, e.g., 36 hours. At the conclusion of the viral infection phase, the cell suspension is harvested. The medium is tested by ELISA assay for the presence of CCL21 chemokine. Vials of cells are stored in a liquid nitrogen freezer.

### Reducing the Viability of Bystander and Cancer Cells

In order to reduce the risk that the cell injected as part of the cell-based vaccine described herein will lead to secondary cancer in the subject, e.g., vaccination site tumors, the cells are treated to reduce their viability, i.e., to induce apoptotic processes. The treated cells will then fragment and undergo apoptosis. In some embodiments, the cells are treated by being irradiated before use, e.g., with 15,000 rads, e.g., from a ¹³⁷Cs source discharging 800 rad/min. In some embodiments, the cells are also subjected to at least one freeze-thaw cycle, e.g., including freezing in liquid nitrogen (−210° C.).

### Preparing a Cell-Based Vaccine Composition

In general, before treatment, the bystander cells and the target cancer cells are thawed, washed, and combined to create a vaccine composition. For example, one vial each of the cells are thawed rapidly by immersion in a 37° C. waterbath, diluted in sterile saline for 15-30 min at 37° C., centrifuged, and resuspended in a final volume of about 1 mL of sterile saline to generate the reconstituted vaccine. In general, the cells will already have been gene transfected or otherwise genetically-modified to express GM-CSF, CD40 ligand, and CCL21. The exact ratio of cells is not crucial, and optimal ratios can be determined based on animal and in vitro studies; for most purposes, roughly equivalent numbers of cells will be sufficient. In some embodiments, additional ingredients can be added to the reconstituted vaccine, e.g., adjuvants.

### Treating Cancer Using Cell-Based Vaccines

The methods described herein include methods for the treatment of cancer. Generally, the methods include administering a therapeutically effective amount of therapeutic agent as described herein, to a subject who is in need of, or who has been determined to be in need of, such treatment. As used herein, the term “treat” means to decrease the growth or growth rate of a tumor, prevent or delay re-growth of a tumor, e.g., a tumor that was debulked, e.g., surgically debulked, or treated using radiation or chemotherapy, or to decrease the size of a tumor. The methods of treatment include initiating or enhancing an anti-tumor immune response in the subject.

As used herein, the term “cancer” refers to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. In general, a cancer can be associated with the presence of one or more tumors, i.e., abnormal cell masses. The term “tumor” is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth.

Tumors include malignancies of the various organ systems, such as affecting lung, breast, thyroid, lymphoid, neural, gastrointestinal, and genito-urinary tract tissues, as well as adenocarcinomas which include malignancies such as most colon cancers, renal cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is colorectal cancer, pancreatic cancer, esophageal cancer, renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation, e.g., soft tissue and bone sarcomas. Malignancies of neural tissues include gliomas and neuroblastomas.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. For example, the diseases can arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin's lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

In some embodiments, cancers treated by the methods described herein include those that are particularly immunogenic, e.g., neuroblastoma, melanoma and renal cell cancer. In other embodiments, cancers treated include lymphoma, non-Hodgkin's lymphoma, leukemia, myeloma, glioma, lung cancer, liver cancer, breast cancer, prostate cancer, gastric cancer, pancreatic cancer, colon cancer, soft tissue sarcoma and bone sarcoma.

The vaccines described herein can be administered to a subject, e.g., a cancer patient, by a variety of routes. For example, subcutaneous, intradermal, or subdermal.

Data obtained from in vitro cell cultures and animal models can be used to project an efficacious dose regimen in humans, including dose and frequency. A projected optimal human efficacious dose regimen can be selected and further tested in clinical trials.

In general, efficacious dose regimen (dose and frequency) ranges for the vaccine include amounts sufficient to treat cancers. Such doses include, e.g., about 1×10⁵ to 1×10⁸ cells per dose, e.g., about 0.5×10⁶ to 1×10⁷ cells per dose, e.g., about 1×10⁶ cells per dose. These numbers are general guidelines, which one of skill in the art can use to determine optimal dosing. Suitable dose frequencies include, e.g., every week for 12 doses, every other week for 6 doses, every 4 weeks for 3 doses, every 3 months. In some embodiments, several doses are administered once every 2 weeks, and then additional doses are administered once a month or once every 3 months. The treatment can also be resumed after a certain period if needed. The dose regimen, including both dose and frequency, can be adjusted based on the genetic, demographic, and pathophysiological characteristics of the subject, and disease status. For example, the age, sex, and weight of a subject to be treated, and the type or severity of the subject's cancer. Other factors that can affect the dose regimen include the general health of the subject, other disorders concurrently or previously affecting the subject, and other concurrent treatments.

The dose of vaccine can be flat (e.g., in cells/dose) or individualized (e.g., in cells/kg or cells/m² dose) based on the safety and efficacy of the treatment and the condition of the subject. The dose and frequency can also be further individualized based on the tumor burden of the subject (e.g., in cells/tumor size, cells/kg/tumor size or cells/m²/tumor size dose). It should also be understood that a specific dose regimen for any particular subject can depend upon the judgment of the treating medical practitioner. In determining the effective amount of the cells to be administered, the treating medical practitioner can evaluate factors such as adverse events, and/or the progression of the disease.

### Combination Therapy

The vaccines described herein can be used as a monotherapy or as part of a combination therapy. For example, the vaccines can be administered to a subject in combination with other treatment modalities with different mechanisms of action, for example, surgery, radiation, cytotoxic chemotherapy (e.g., cyclophosphamide, 5-fluorouracil, cisplatin, gemcitabine), targeted biologic agents (e.g., monoclonal antibodies, fusion proteins), and immune modulators (e.g., cytokines and/or CTLA-4, PDL-1, PD-1 antibodies). These combination therapies can have additive or synergistic effects. The vaccines can also be used in combination with other cancer vaccines that carry different tumor-associated antigens. The various treatments can be administered concurrently or sequentially (e.g., before or after treatment using a method described herein). For example, one treatment can be given first, followed by the initiation of administration of other treatments after some time. A previous therapy can be maintained until another treatment or treatments have effect or reach an efficacious level.

For example, a surgical treatment method is administered first, to remove as much of the tumor tissue as possible, and then one or more doses of a vaccine as described herein are administered. In another example, one or more doses of a vaccine as described herein are administered prior to administration of a dose of cytotoxic radiation or chemotherapy, e.g., to sensitize the tumor cells to the radiation or chemotherapy and thereby enhance the effect of the radiation or chemotherapy. Thus, the methods described herein can include first administering one or more doses of a vaccine as described herein, followed by one or more doses of radiation or chemotherapy.

In some embodiments, the subjects are administered all trans retinoic acid (ATRA), e.g., before beginning vaccine administration and optionally again after the first 1, 2, 3, 4, 5 or more doses of vaccine. ATRA has been shown to improve the ratio of DC to immature myeloid cells (ImC) in cancer patients and in pre-clinical models (Almand et al., J Immunol 166:678 (2001); Kusmartsev et al., Cancer Res 63:4441 (2003)). ATRA is commercially available, e.g., as tretinoin, trade name VESANOID™ manufactured by Roche. An exemplary dose is 150 mg/m²/d.

Cyclophosphamide has been used as a tumor vaccine augmentation strategy in clinical trials (Berd et al., J Clin Oncol 22(3):403 (2004); Berd et al., Int J Cancer 94(4):531 (2001)), and the mechanism of this effect has recently been shown to be by decreasing the number and function of regulatory T cells (T reg, naturally occurring suppressor T cells). In some embodiments, subject will be administered one or more doses of cyclophosphamide, e.g., after or concurrently with the ATRA dose and prior to the first dose of vaccine. Cyclophosphamide is commercially available, e.g., from Bristol Meyers Squibb. An exemplary dose of cyclophosphamide is 300 mg/m². As this dose has moderate emetogenic potential, ondansetron 16 mg PO and lorazepam 1 mg IV can be administered, e.g., prior to chemotherapy infusion.

### Evaluating Subjects Pre-Treatment and Post-Treatment

Prior to initiation of the vaccine treatment, subjects can be tested for the need of treatment. The clinical signs and symptoms of cancer, which are known in the art, can be an indicator of treatment need although an earlier predictor of treatment is more desirable. The dose regimen of the vaccine can be adjusted based on the severity of clinical signs and symptoms of cancer.

Following administration of a vaccine as described herein, the efficacy and safety of the treatment can be assessed in several ways, indirectly or directly. The parameters, including levels of biomarkers (for example, immune responses such as the presence of reactive T cells, increased IFN-γ production), clinical signs and symptoms (for example, tumor lesions (e.g., growth and/or overall size) by imaging or clinical measurements, response rate, time to progression, progression-free survival, or overall survival), and adverse events, can be evaluated over time in the same subject. The parameters can also be compared between actively treated subjects and placebo subjects at the same time points. The parameters can be the absolute values or the relative changes from the baseline in the same subject or compared to placebo subjects. The levels of biomarkers associated with cancer and treatment in subject samples can be monitored before and after treatment. The number and/or severity of clinical signs and symptoms in a subject can be compared before and after treatment, including long-term follow-up after the last dose. The adverse events can also be monitored and compared between active and placebo groups or between baseline and post-treatment in the active group. For example, a subject (e.g., a cancer patient) can have an initial assessment of the severity of his or her disorder (e.g., the number or severity of one or more symptoms of cancer), receive vaccine treatment as a monotherapy or part of a combination therapy, and then be assessed subsequently to the treatment at various time points (e.g., at one day, one week, one month, three months, six months, one year, two years and three years). See e.g., Example 5, herein.

### EXAMPLES

The present invention is further described in the following examples, which do not limit the scope of the invention described in the claims.

### Example 1

### Preparation of a GM.CD40L.CCL21 Vaccine

An exemplary cell-based vaccine was prepared comprising a bystander cell line genetically-modified to stably express GM-CSF and CD40 Ligand, with lung cancer cells from two different cell lines, one of which was gene-modified to express CCL21.

### Bystander Cell Line Genetically-Modified to Express GM-CSF and CD40 Ligand

The bystander cell line genetically-modified to express GM-CSF and CD40 ligand was prepared by methods known in the art. See, e.g., Dessureault et al., J Surg Res 125:173 (2005); Dessureault et al., Ann Surg Oncol 14(2):869 (2006); U.S. patent application Ser. Nos. 10/620,746 and 12/173,514.

The GM.CD40L bystander cell line was established by transfecting the human erythroleukemia K562 cell line with the cDNAs for hGM-CSF and hCD40L. Like the K562 parent cell line from which it is derived, the GM.CD40L bystander cell line is an MHC-negative cell line that grows in cell suspension.

The cDNA for human CD40L was first excised from the pcDL-SRalphahCD40L cloning vector (ATCC #79814) using a BamHI restriction digest, and then inserted into the multiple cloning site of the expression vector pNGVL3 (gift of Dr. Gary J. Nabel, NIH; also available from the National Gene Vector Laboratory, Ann Arbor, Mich.), which contains the gene for kanamycin resistance. Restriction enzyme digest analysis confirmed appropriate release of the isolated hCD40L cDNA. The correct reading frame was confirmed by in-line sequencing of the hCD40L gene in the pNGVL3 plasmid.

K562 cells were transfected with the pNGVL3hCD40L plasmid by electroporation. Briefly, K562 cells in log phase growth were harvested, washed twice with PBS, resuspended at 1×10⁷ cells per mL, and transferred to electroporation cuvettes (BTX, Genetronics Inc., Model #640) on ice. Plasmid DNA (40 mg) was added to the cell suspension and incubated on ice for 5 min. The mixture was then electroporated with 250 volts at a capacitance setting of 960 mF. The cuvettes were kept at room temperature for 5 min, then the transfected cells were diluted 1:20 in nonselective Iscove's complete medium and incubated for culture. Cells were sorted by flow cytometry three times for CD40L expression, followed by cloning by limiting dilution of the cell pool. The final positive clone was grown in culture and frozen for future usage.

The singly transduced K562-CD40L cells described above was transfected with the pCEP4hGM-CSF construct (gift of Ivan Borrello, Johns Hopkins University) containing the hGM-CSF gene (505 bp) and the gene for Hygromycin B resistance. Briefly, the plasmid DNA was digested with BlnI and ClaI restriction enzymes overnight at 37° C. and run on a 1% Seakam agarose gel at 100 volts. The linearized band was cut from the gel and purified by the Freeze and Squeeze method. DMRIE-C Reagent (GIBCO, Life Technologies, Cat #10459-014) was used to deliver the linearized plasmid into the K562 and K562-CD40L cells. (This reagent is a 1:1 (M/M) liposome formulation of the cationic lipid DMRIE and cholesterol in membrane-filtered water. The positively charged and neutral lipids form liposomes that can complex with nucleic acids.) Hygromycin B (500 mg/mL) was added to the cultures after 48 hours and resulting colonies were transferred to 96-well tissue culture plates after 10 days. Subsequent clones were grown in 24-well tissue culture plates and tested for GM-CSF production by ELISA. Positive clones were identified, grown in culture, and frozen for future usage. A stable transfected clone was designated K562-GM-CSF-CD40L.

The K562-GM-CSF-CD40L clone used for generation of the Master Cell Bank (clone #1) has been named “GM.CD40L” and will be used and distributed under this name. Once the transfected cell line was established (March 2001), medium in which cells were propagated was converted to AIM-V serum-free medium (Life Technologies, GIBCO BRL, Catalog #12055-091). All subsequent cell passages were carried out in this medium (supplemented with hygromycin B).

A Master Cell Bank (MCB) was generated by serial subculture and expansion of the original GM.CD40L clone until 4×10⁸ cells were available for simultaneous harvest and cryopreservation. This created a uniform population of cells which was divided equally into 19 vials (2×10⁷ cells per vial) and stored in the vapor phase of liquid nitrogen. The Manufacturer's Working Cell Bank (MWCB) was generated from two ampoules of the MCB. The MCB source cells (passage 8) were thawed and expanded by serial subculture in AIM-V serum-free medium (Life Technologies, GIBCO BRL) containing hygromycin B (500 mg/mL) (Sigma Aldrich, St. Louis, Mo., USA). Cells were removed from hygromycin B-containing medium and returned to fresh AIM-V serum-free medium 48 h prior to final harvest for the MWCB. The viability of these harvested cells, as determined by trypan blue exclusion, was 83%. A fraction of the cells was dispensed into 48 individual ampoules (2×10⁷ cells per ampoule), and cryopreserved to form the MWCB. Another fraction of the cells was irradiated (15,000 rad) and dispensed into 81 ampoules (5×10⁶ cells per ampoule), and cryopreserved to form the first lot (L001) of the biological vaccine product. All subsequent lots (L002, L003, L004, and so on) were generated from single ampoules of the MWCB.

### Target Cancer Cells Genetically-Modified to Express CCL21

A mixture of two human non-small cell lung cancer (NSCLC) cell lines served as the source of lung tumor antigens. These cell lines, NCI-H1944 and NCI-H2122, combined express the following tumor antigens that are commonly over-expressed in NSCLC: HER-2/neu, CEA, GD-2, WT-1, and MAGE-1, -2 and -3 (Wroblewski et al., Lung Cancer 33:181 (2001)). These cell lines were obtained from ATCC. The H1944 cell line was transduced with CCL21 cDNA.

CCL21 gene bearing adenovirus (Ad.CCL21) was obtained from the Cancer Institute (NCI) Rapid Access to Intervention Development program (RAID) program at the Cancer Therapy Evaluation Program (CTEP) of NCI. The generation, storage, characterization, production, and quality control testing of the H1944 cell line combination with Ad.CCL21 were performed according to the standard operating procedures at the Cell Therapy Core of the Moffitt Cancer Center.

The H1944 cell line was maintained in a MWCB. Individual lots were grown as described for GM.CD40L cells until the desired cell number was achieved. Cells were harvested from the flasks, centrifuged, and resuspended in 50 mL conical tubes to a cell concentration of 10⁸ cells per mL. CCL21 gene bearing adenovirus (Ad.CCL21) was added to the tubes at an MOI of 10,000 pfu/cell. Tubes are placed upright in a 37° C. incubator for 2 hours to promote viral adsorbtion to cells. Following the 2-hour incubation, cells were adjusted to 1.5×10⁷ cells/mL, returned to flasks, and returned to the incubator for 24 additional hours. At the conclusion of the viral infection phase, the cell suspension was harvested as with the other cell lines. The medium was tested by ELISA assay for the presence of CCL21 chemokine. Vials of cells were stored in a liquid nitrogen freezer.

### GM. CD40L. CCL21 Vaccine

On the day of treatment, a vial of each cell type, including (1) the bystander cell line expressing GM-CSF and CD40 ligand; (2) a first population of target cancer cells; and (3) a second population of target cancer cells expressing CCL21, was thawed, washed, and combined in sterile saline with the cell equivalent ratio of about 1:1:1 to create the final vaccine composition.

### Example 2

### CCL21 Secretion by NSCLC H1944 Transduced with Ad.CCL21

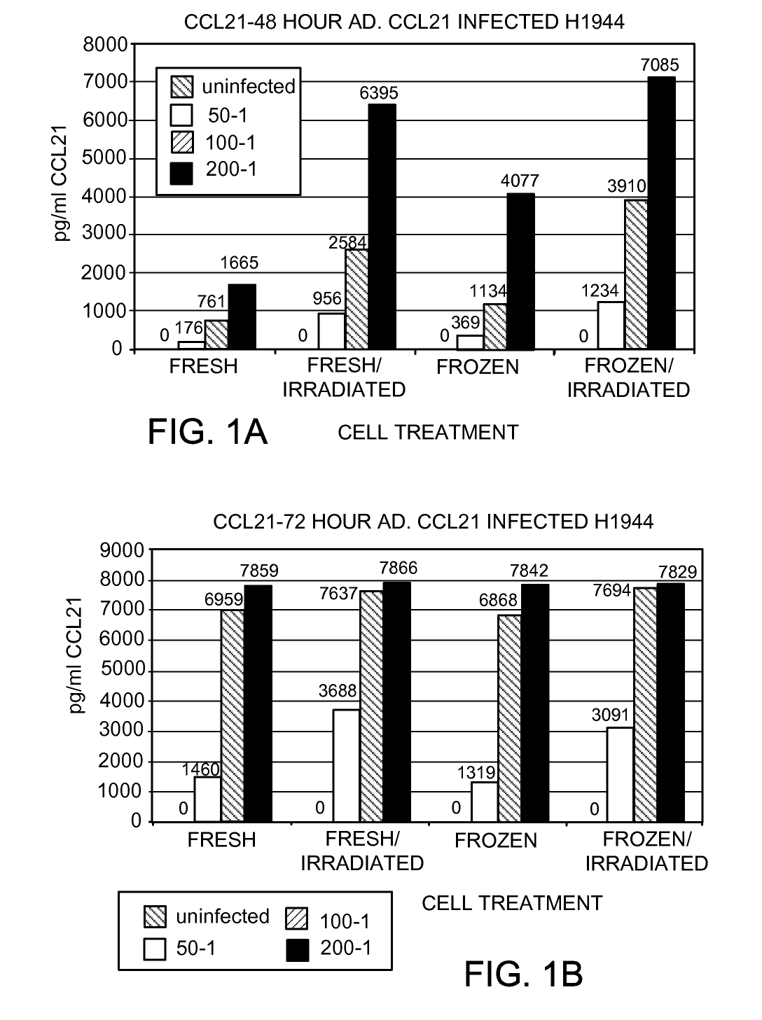

The human lung adenocarcinoma cell line H1944 was infected with a recombinant adenoviral vector containing the human CCL21 cDNA as described above. Aliquots were radiated with 15,000 rads from a ¹³⁷Cs source discharging 800 rad/min, frozen and thawed, then placed into culture. The culture supernatants were assayed for the presence of CCL21 in ELISA assays at 48 hours (FIG. 1A) and 72 hours (FIG. 1B). The concentrations of CCL21 secreted in the culture supernatants increased in a multiplicity of infection (MOI)- and time-dependent manner Neither freezing-thawing nor irradiation had any effect on CCL21 expression.

### Example 3

### H1944-Derived CCL21-Induced T Cell Migration

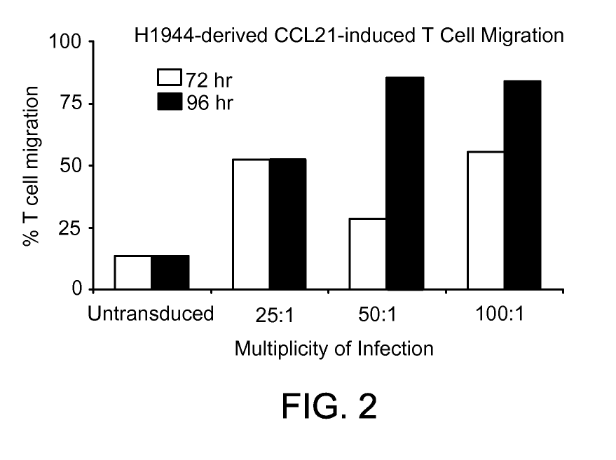

Naïve T cells migrate in response to CCL21 secreted by Ad.CCL21-transduced H1944 cells (TM.CCL21). Naïve T cells were obtained from PBMC of a healthy donor using an untouched T-cell isolation kit. Chemotaxis was assayed at 72 and 96 hours after transduction using Corning Transwell plates in a standard chemotaxis assay (Siegmund, “Chemotaxis Assay: Analysis of Migration of Lymphocyte Subsets,” in Leukocyte Trafficking, Hamann, Editor. 2006. pp. 418-423). CCL21 secretion at various multiplicities of infection (MOI) was measured by ELISA of cell culture supernatants at various time points. Infection of cells with virus particles can induce cell death in some cell lines. To minimize transduction-associated cell death, the lowest ratio of virus particle to tumor cell would be optimal. At 50:1 MOI, nearly 5 ng/mL CCL21 were secreted by TM.CCL21 cells in 96 hours. Although the level of CCL21 production increased with higher MOI, T cell migration did not improve significantly, indicating that 50:1 MOI may be optimal for Ad.CCL21 transduction of this particular cell line (FIG. 2).

### Example 4

### CCL21 Effects on IL-2 Production

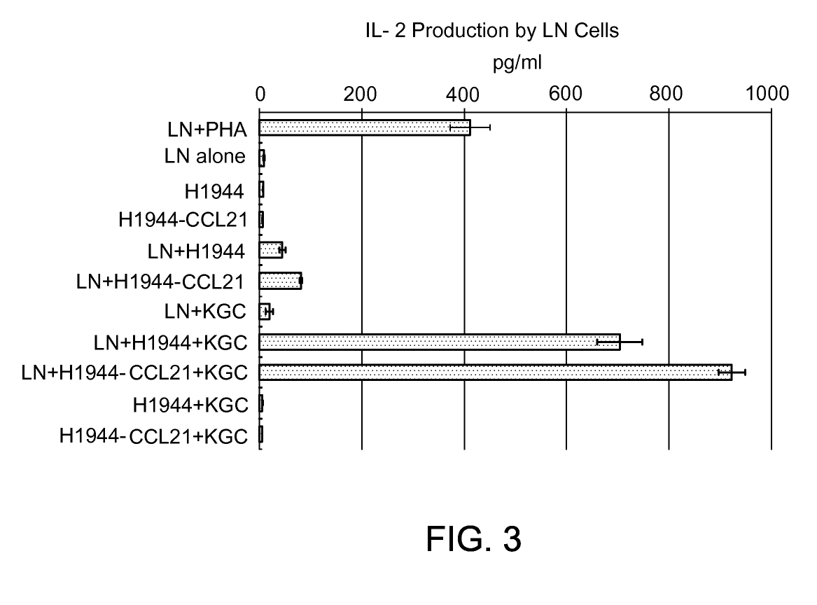

CCL21 expression augmented anti-tumor immune responses induced by GM.CD40L-transfected bystander cells. Lymph node (LN) cells co-cultured in the presence of GM.CD40L and TM.CCL21 enhanced immune responses, as measured by T cell-associated IL-2 production over un-transduced H1944 tumor cell line. Although TM.CCL21 slightly increased IL-2 secretion by lymph node cells, the presence of both GM.CD40L and TM.CCL21 were necessary to promote a robust anti-tumor response (FIG. 3). Consequently, the combination of all components of GM.CD40L.CCL21 vaccine may significantly improve the tumor-specific immune responses and clinical efficacy in cancer patients.

### Example 5

### Clinical Study of GM.CD40L.CCL21 Vaccine

Patients are randomized to one of two arms (ratio is 1:1) of GM.CD40L versus GM.CD40L.CCL21. Intradermal vaccine injections at four separate sites near lymph nodes (bilateral upper arms and bilateral upper thighs) are performed every 14 days times 3 followed by every 28 days times 3 (on days 1, 14, 28, 56, 84, and 112). Vaccine A consists of GM.CD40L cells admixed with an equivalent number of allogeneic tumor cells, whereas vaccine B consists of GM.CD40L cells admixed with an equivalent number of allogeneic tumor cells expressing CCL21.

Patients are monitored for evidence of toxicity and the development of a specific immune response. Patients who are found to have stable disease (SD), partial response (PR), or complete response (CR) at re-staging after the initial 6 vaccine doses may receive additional vaccines every 3 months until evidence of disease progression. Patients are followed for the rest of their lives. Overall survival and time to progression are also determined.

Response and progression are evaluated using the international criteria proposed by the Response Evaluation Criteria in Solid Tumors (RECIST) Committee. Changes in only the largest diameter (unidimensional measurement) of the tumor lesions are used in the RECIST criteria. Note: Lesions are either measurable or non-measurable using the criteria provided below. The term “evaluable” in reference to measurability will not be used because it does not provide additional meaning or accuracy.

Measurable Disease. Measurable lesions are defined as those that can be accurately measured in at least one dimension (longest diameter to be recorded) as >20 mm with conventional techniques (CT, MRI, x-ray) or as >10 mm with spiral CT scan. All tumor measurements will be recorded in millimeters (or decimal fractions of centimeters).

Non-Measurable Disease. All other lesions (or sites of disease), including small lesions (longest diameter <20 mm with conventional techniques or <10 mm using spiral CT) are considered non-measurable disease. Bone lesions, leptomeningeal disease, ascites, pleural/pericardial effusions, lymphangitis cutis/pulmonis, abdominal masses (not followed by CT or MRI) and cystic lesions are non-measurable.

Target Lesions. All measurable lesions up to a maximum of five lesions per organ and 10 lesions in total representative of all involved organs will be identified as target lesions and recorded and measured at baseline. Target lesions will be selected on the basis of their size (lesions with the longest diameter) and their suitability for accurate repeated measurements (either by imaging techniques or clinically). A sum of the longest diameter (LD) for all target lesions will be calculated and reported as the baseline sum LD. The baseline sum LD will be used as reference by which to characterize the objective tumor response.

Non-Target Lesions. All other lesions (or sites of disease) should be identified as non-target lesions and will also be recorded at baseline. Non-target lesions include measurable lesions that exceed the maximum numbers per organ or total of all involved organs as well as non-measurable lesions. Measurements of these lesions are not required, but the presence or absence of each should be noted throughout follow-up.

Evaluation of Measurable Disease. All measurements should be taken and recorded in metric notation using a ruler or calipers. All baseline evaluations will be performed as closely as possible to the beginning of treatment and never more than 4 weeks before the beginning of the treatment. The cytological confirmation of the neoplastic origin of any effusion that appears or worsens during treatment when the measurable tumor has met criteria for response or stable disease is mandatory to differentiate between response or stable disease (an effusion may be a side effect of the treatment) and progressive disease.

Response Criteria.

### Evaluation of Target Lesions.

Complete Response (CR): Disappearance of all target lesions.
Partial Response (PR): At least a 30% decrease in the sum of the LD of target lesions, taking as reference the baseline sum LD.
Stable Disease (SD): Neither sufficient shrinkage to qualify for PR nor sufficient increase to qualify for PD, taking as reference the smallest sum LD since the treatment started.
Progressive disease (PD): At least a 20% increase in the sum of the LD of target lesions, taking as reference the smallest sum LD recorded since the treatment started or the appearance of one or more new lesions.

### Evaluation of Non-Target Lesions.

Complete Response (CR): Disappearance of all non-target lesions.
Stable Disease (SD): Persistence of one or more non-target lesion(s).
Progressive Disease (PD): Appearance of one or more new lesions and/or unequivocal progression of existing non-target lesions.

### Other Embodiments

It is to be understood that while the technology has been described in conjunction with the detailed description thereof, the foregoing description is intended to illustrate and not limit the scope of the technology, which is defined by the scope of the appended claims. Other aspects, advantages, and modifications are within the scope of the following claims.

1. A population of cells that have been genetically-modified to express exogenous macrophage colony stimulating factor (GM-CSF), exogenous CD40 ligand (CD40L), and exogenous chemokine C—C motif ligand 21 (CCL21), wherein the population of cells comprises bystander cells and target cancer cells.

2. The population of cells of claim 1, wherein the bystander cells express GM-CSF and CD40L.

3. The population of cells of claim 2, wherein the bystander cells also express CCL21.

4. The population of cells of claim 1, wherein the target cancer cells express CCL21.

5. The population of cells of claim 1, wherein the bystander cells are major histocompatibility complex (MHC) negative.

6. The population of cells of claim 1, wherein the bystander cells are from the cell line K562.

7. The population of cells of claim 1, wherein the target cancer cells comprise cells from a solid or hematopoietic-derived tumor.

8. The population of cells of claim 1, wherein the target cancer cells comprise cells from allogeneic cancer cell lines or autologous cancer cells.

9. The population of cells of claim 1, wherein the target cancer cells comprise cells from two or more different cancer types or different cell lines.

10. The population of cells of claim 9, wherein the different cancer cell lines comprise cells from two or more different human lung adenocarcinoma cell lines.

11. The population of cells claim 1, wherein the cells have been treated to reduce cell viability.

12. A therapeutic composition for inducing an immune response to a cancer in a subject, the composition comprising the population of cells of claim 11.

13. A method of preparing a population of cells for use in a therapeutic composition, the method comprising:
providing a population of cells according to claim 1; and
treating the cells to reduce cell viability.

14. (canceled)

15. (canceled)

16. (canceled)

17. A method of treating a cancer in a subject, the method comprising:
administering to the subject a therapeutically effective amount of a composition comprising a population of cells according to claim 11.

18. The method of claim 17, wherein the target cancer cells comprise cancer cells that are autologous to the subject to be treated.

19. The method of claim 17, wherein the target cancer cells comprise cells from a cancer of the same type as the cancer in the subject.

20. The method of claim 17, wherein the target cancer cells comprise cells from a cell line made from cells of a cancer of the same type as the cancer in the subject.

21. The method of claim 17, wherein the cancer is selected from the group consisting of: lymphoma, non-Hodgkin's lymphoma, leukemia, myeloma, glioma, neuroblastoma, lung cancer, kidney cancer, liver cancer, breast cancer, prostate cancer, gastric cancer, pancreatic cancer, colon cancer, soft tissue sarcoma, bone sarcoma and melanoma.

22. The method of claim 17, wherein the subject is a non-human animal or a human.

23. The method of claim 17, wherein the composition is administered by a route of administration selected from the group consisting of: subcutaneous, intradermal and subdermal.

24. The method of claim 17, further comprising administering one or more additional treatments to the subject.

25. The method of claim 17, further comprising administering one or more additional doses of the composition.

26. The method of claim 17, further comprising identifying a subject having a cancer.

27. The method of claim 17, further comprising monitoring the subject for one or more clinical parameters of cancer.

28. The method of claim 27, wherein the one or more clinical parameters of cancer are selected from the group consisting of: tumor growth, tumor regrowth and survival.
